(19) United States
(12) Patent Application Publication
(10) Pub. No.: US 2026/0159858 A1
(43) Pub. Date: Jun. 11, 2026

(54) MODIFIED DRY GRIND METHOD AND SYSTEM FOR BIOCHEMICAL AND/OR BIOFUEL PRODUCTION

(71) Applicant: Fluid Quip Technologies, LLC, Cedar Rapids, IA (US)

(72) Inventor: Neal Jakel, Cedar Rapids, IA (US)

(21) Appl. No.: 19/401,462

(22) Filed: Nov. 26, 2025

Related U.S. Application Data
(60) Provisional application No. 63/728,978, filed on Dec. 6, 2024.

(51) Int. Cl. C12P 7/06

(52) U.S. Cl. CPC C12P 7/06

(57) ABSTRACT

A modified dry grind method and system for biochemical and/or biofuel production, such as alcohol, in which dried grain (e.g., corn) feedstock is initially wetted prior to being milled to reduce the complexity and the amount of electricity typically consumed in the grain handling and milling part of a dry grind process. In one example, the method and system can include mixing a grain feedstock with a liquid in a holding tank to initially wet the grain. Then, the mixture can be subjected to a milling step in which the grain can be broken down, such as by a disc mill, into smaller ground pieces. Prior to being milled, the mixture of wet corn and liquid can be subjected to a dewatering device to remove liquid from the wet grain. Once milled, the milled grain can be further subjected to traditional, modified traditional, or non-traditional dry grind steps to produce biofuel and/or biochemicals.

### TECHNICAL FIELD

The present invention relates generally to dry grind methods and systems for biochemical and/or biofuel production, such as alcohol (e.g., ethanol).

### BACKGROUND

Dry milling ethanol plants generally convert corn and/or other grains into three products, i.e., ethanol, distillers grain oil, and distiller's grains with solubles. A typical grain dry milling process consists of four major steps: grain handling and dry milling, liquefaction and saccharification, fermentation and distillation, and co-product recovery. Grain handling and dry milling is the step in which the grain is brought into the plant and ground to promote better conversion of starch to glucose. Liquefaction is the step of converting solids, such as starch, to a flowable liquid producing oligosaccharides and saccharification is where the oligosaccharides are converted into single glucose molecules. Fermentation is the process of yeast or bacteria, or as clostridia, for example, converting glucose into a biofuel or a biochemical/biomolecule, such as ethanol. Distillation is the process of removing the biofuel or biochemical/biomolecule, such as ethanol, from the fermentation product. Co-product recovery is the step in which the grain by-products are de-watered and made ready for market. There are many known chemical and biological conversion processes known in the art that utilize yeast, bacteria, or the like to convert glucose/sugar to other biofuels and biochemical/biomolecule components like ethanol, for example.

The recovery of alcohol, e.g., butanol, ethanol (a natural co-product), etc., and other similar compounds, generally begins with the beer (spent fermentation broth) being sent to a distillation system. With distillation, ethanol is typically separated from the rest of the beer through a set of stepwise vaporizations and condensations. The beer less the alcohol extracted through distillation is known as whole stillage, which contains a slurry of the spent grains including grain protein, fiber, oil, minerals, sugars, and fermentation agent. This byproduct is too diluted to be of much value at this point and is further processed to provide the distiller's grains with solubles.

In typical processing, when the whole stillage leaves the distillation column, it is generally subjected at the back end of the process to a decanter centrifuge to separate insoluble solids or “wet cake”, which includes mostly fiber, from the liquid or “thin stillage”, which includes, e.g., protein, fine fiber, oil, and amino acids. After separation, the thin stillage moves to evaporators to boil away moisture, leaving a thick syrup that contains the soluble (dissolved) solids. The concentrated syrup is typically mixed with the wet cake, and the mixture may be sold to beef and dairy feedlots as distillers wet grain with solubles (DWGS). Alternatively, the wet cake and concentrated syrup mixture may be dried in a drying process and sold as distillers dried grain with solubles (DDGS). The resulting DDGS generally has a crude protein content of about 29% and is a useful feed for cattle and other ruminants due to its protein and fiber content.

Specifically concerning the grain handling and milling of the dried grain (e.g., corn) feedstock in a dry grind process, an undesirable amount of electricity can be consumed when all of the various auxiliary equipment that is needed to not only grind, but mitigate dust, and then convey the resulting ground grain to the next step(s) in the method and system is taken into consideration. Additionally, when dry grain is ground there is little control of the particle size distribution as the typical processes use more of a brute force to shatter the grain into smaller particles, resulting in a wide range of ground particles. The dry milling process can yield very fine grain particles (<45 microns) to larger essentially intact particles in the size of 2,000 microns. This wide variation of particles can be challenging to process through the system resulting in higher enzymes costs, more equipment needed, poor conversions, and other inefficiencies throughout the typical grain process.

Accordingly, it would be beneficial to provide a method and system for biochemical and/or biofuel production that can reduce the complexity and the amount of electricity typically consumed in the grain handling and milling part of a dry grind process as well as reduce the variability of the ground grain particles, thereby helping to keep overall capital and operational costs at a desirable level for the dry grind plant.

### SUMMARY OF THE INVENTION

The present invention is directed to a modified dry grind method and system for biochemical and/or biofuel production, such as alcohol (e.g., ethanol), in which dried grain (e.g., corn) feedstock initially is wetted or hydrated prior to being milled.

In one embodiment, the modified dry grind method and system can include mixing a grain feedstock (e.g., corn) with a liquid (e.g., water and/or backset) in a holding tank for about 3 minutes to about 30 minutes, for example, to initially wet or moisten the grain (e.g., corn kernels). Then, the mixture of wet corn and liquid can be sent and subjected to a milling step in which the grain can be broken down, such as by a disc mill or the like, into smaller ground pieces or particles. One or more enzymes and/or chemicals can be added after the holding tank to aid in starch reductions as well as to facilitate grain breakage/milling of the grain. Prior to being milled, the mixture of wet corn and liquid can optionally be subjected to a dewatering device, which can include a screen (e.g., a gravity screen), to remove a liquid portion from the wet grain. In one example, the liquid provided to the holding tank can be devoid of enzyme(s) and/or other chemical processing aides, such as sulfur dioxide. Once milled, the milled grain can be subjected to traditional or non-traditional dry grind steps to produce biofuel and/or biochemicals.

This initial wetting of the corn or wet milling process, which provides for a modified dry grind method and system for biochemical and/or biofuel production, is understood to differ from traditional wet milling in a wet mill plant in that a traditional wet mill is generally focused on softening/soaking the entire grain/kernel (e.g., corn) throughout the internal of the grain (e.g., corn kernel) so that germ, for example, can be removed whole and intact in a first grind mill. In contrast, this wet milling is not concerned with maintaining a whole germ or to have liquid/water migration into the internal of the kernel, so there is no need to soak the kernel for an extended period of time, such as for 24 or more hours, or soak with an enzyme or chemical aide, such as sulfur dioxide. Instead of initially dry milling the grain feedstock, which can consume an undesirable amount of electricity due to all of the various auxiliary equipment that is needed to not only grind, but mitigate dust, and then convey the resulting ground grain to the next step(s), the wetting of the corn/wet milling process can help keep overall capital costs at a desirable level for a dry grind plant. Additionally, the traditional wet milling process typically requires whole intact corn kernels, whereas this modified process can utilize broken or cracked grain/kernels.

### BRIEF DESCRIPTION OF THE DRAWINGS

The accompanying drawings, which are incorporated in and constitute a part of this specification illustrate embodiments of the invention and, together with the general description of the invention given above and the detailed description given below, serve to explain the principles of the invention. Similar reference numerals are used to indicate similar features throughout the various figures of the drawings.

FIG. 1 is a flow diagram of a typical dry grind alcohol production process; and

FIG. 2 is a flow diagram of a modified dry grind method and system for biochemical and/or biofuel production in which dried grain feedstock is initially wetted prior to milling in accordance with an embodiment of the invention.

### DETAILED DESCRIPTION OF SPECIFIC EMBODIMENTS

The present invention relates to a modified dry grind method and system for biochemical and/or biofuel production, such as alcohol (e.g., ethanol), in which dried grain (e.g., corn) feedstock initially is wetted prior to being milled. In particular, in a typical grain dry milling, dried grain is brought into a dry grind plant and ground to promote conversion of starch to glucose in an initial grain handling and milling step. But, with the grain handling and milling of the dried grain (e.g., corn) feedstock, an undesirable amount of electricity can be consumed when all of the various auxiliary equipment that is needed to not only grind, but mitigate dust, and then convey the resulting ground grain to the next step(s) in the dry grind process is taken into consideration. Here, the dried grain (e.g., corn) feedstock or, alternatively, an already elevated moisture grain (e.g., corn) feedstock with a moisture content typically found in field harvested grain (e.g., 20-24% moisture or higher) initially is wetted prior to being milled, which provides for a modified dry grind method and system for biochemical and/or biofuel production, as further discussed in detail hereinbelow, so as to reduce the amount of electricity typically consumed in the grain handling and milling part of a dry grind process thereby helping to keep overall capital costs at a desirable level for the dry grind plant.

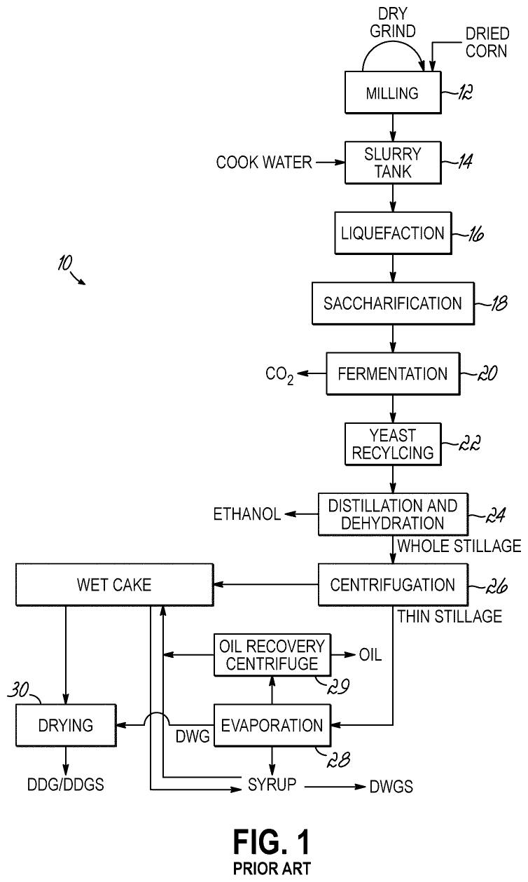

With reference now to FIG. 1, this figure shows a flow diagram of a typical dry grind alcohol (e.g., ethanol) production method 10. Although virtually any type and quality of grain, such as but not limited to sorghum, wheat, triticale, barley, rye, tapioca, cassava, potato, pea and other starch and/or oil containing grains and/or legumes can be used to produce ethanol and/or a biochemical/biomolecule, for example, the feedstock for this process is typically corn referred to as “No. 2 Yellow Dent Corn.” Also, as a general reference point, the dry grind method 10 can be divided into a front end and a back end. The part of the method 10 that occurs prior to distillation 24 is considered the “front end,” and the part of the method 10 that occurs after distillation 24 is considered the “back end.” To that end, the front end of the dry grind method 10 begins with a milling step 12 in which dried whole corn kernels can be passed through hammer mills for grinding/milling into meal or a fine powder. The screen openings in the hammer mills or similar devices typically are of a size 6/64 to 9/64 inch, or about 2.38 mm to 3.57 mm, but some plants can operate at less than or greater than these screen sizes. The resulting particle distribution yields a very widely spread, bell type curve, which includes particle sizes as small as 45 microns and as large as 2 mm to 3 mm. The majority of the particles tend to be in the range of 500 to 1200 microns, which is the “peak” of the bell curve. Other screen openings larger and smaller can be deployed in the hammer mills. Other milling devices such as roller mills or pin mills can also be utilized in the dry grain grinding step. It is noted that the grain feedstock, which is typically dried down to about 14% moisture, is necessary for a typical hammer mill system. High moisture grain can be milled but becomes more difficult and, thus, large(r) screens in the mills must be used to ensure that the wetter grain can be pulled through the screen. Screen openings typically have to increase to size 9 or 10 ( 9/64″ or 10/64″ openings) to prevent plugging of the wetter grain feedstock material. Moistures of grain (e.g., corn) feedstock generally cannot be processed in a traditional hammer mill much above 24%.

After the milling step 12, the ground meal is mixed with cook water to create a slurry at the slurry tank 14 and a commercial enzyme called alpha-amylase is typically added (not shown). Creating the slurry at the slurry tank 14 is followed by a liquefaction step 16 whereat the pH is adjusted to about 4.8 to 5.8 and the temperature maintained between about 50° C. to 105° C. so as to convert the insoluble starch in the slurry to soluble starch. The stream after the liquefaction step 16 has about 30% dry solids (DS) content, but can range from about 29-36%, with all the components contained in the corn kernels, including starch/sugars, protein, fiber, starch, germ, grit, oil, and salts, for example. Higher solids are achievable, but this requires extensive alpha amylase enzyme to rapidly breakdown the viscosity in the initial liquefaction step. There generally are several types of solids in the liquefaction stream: fiber, germ, and grit.

Liquefaction 16 may be followed by separate saccharification and fermentation steps, 18 and 20, respectively, although in most commercial dry grind ethanol processes, saccharification and fermentation can occur simultaneously. This single step is referred to in the industry as “Simultaneous Saccharification and Fermentation” (SSF). Both saccharification and SSF can take as long as about 50 to 60 hours. Gluco-Amylase enzyme is typically added to the fermentation step that facilitates the further breakdown of the starches and larger polysaccharides into single monomer sugar molecules that the yeast consumes to produce ethanol (or other similar alcohols) and carbon dioxide. Yeast can optionally be recycled in a yeast recycling step 22 either during the fermentation process or at the very end of the fermentation process. Yeast produced during the fermentation process will pass through to the distillation and dehydration step 24. In addition to the gluco-amylase being added, other enzymes can be added (such as but not limited to phytase, protease, cellulase, hemicellulose, xylanase, beta-glucanase, and the like) that can further enhance the protein and oil recovery downstream. Subsequent to the fermentation step 20 is the distillation (and dehydration) step 24, which utilizes a still to recover the alcohol (e.g., ethanol).

Finally, a centrifugation step 26 involves centrifuging the residuals, i.e., “whole stillage”, which includes the non-fermentable grain components (protein, oil, fiber, ash, and minerals, for example) and yeast yielded from the distillation and dehydration step 24 in order to separate the insoluble solids (“wet cake”) from the liquid (“thin stillage”). The liquid from the centrifuge contains about 5% to 12% DS. The “wet cake” includes fiber, of which there generally are three types: (1) pericarp, with average particle sizes typically about 1 mm to 3 mm; (2) tipcap, with average particle sizes about 500 micron; (3) and fine fiber, with average particle sizes of about 250 microns. There may also be proteins and yeast bodies with a particle size of about 45 microns to about 300 microns. The fiber and other fractions may contain bound protein that is chemically and or physically attached to the fiber and other fraction.

The thin stillage typically enters evaporators in an evaporation step 28 in order to boil or flash away moisture, leaving a thick syrup which contains the soluble (dissolved) solids (mainly protein and starches/sugars) from the fermentation (25 to 40% dry solids) along with residual oil and fine fiber. The concentrated slurry can be sent to a centrifuge to separate the oil from the syrup in an oil recovery step 29. The oil can be sold as a separate high value product. The oil yield is normally about 0.9 lb/bu of corn with elevated free fatty acids content compared to traditional wet mill corn oil. This oil yield recovers only about ½ of the oil in the corn, with part of the oil passing with the syrup stream and the remainder being lost with the fiber/wet cake stream. About one-half of the oil inside the corn kernel remains inside the germ after the distillation and dehydration step 24, which cannot be separated in the typical dry grind process using centrifuges as the oil is bound, not free. The free fatty acids content, which is created when the oil is heated and exposed to oxygen throughout the front and back-end process, reduces the value of the oil. The (de-oil) centrifuge only removes less than 50% because the protein and oil make an emulsion, which cannot be satisfactorily separated without the use of chemicals or added mechanical separation unit operations.

The syrup, which has more than 10% oil, can be mixed with the centrifuged wet cake, and the mixture may be sold to beef and dairy feedlots as Distillers Wet Grain with Solubles (DWGS). Alternatively, the wet cake and concentrated syrup mixture may be dried in a drying step 30 and sold as Distillers Dried Grain with Solubles (DDGS) to dairy and beef feedlots. This DDGS has all the corn and yeast protein and about 50% of the oil in the starting corn material. But the value of DDGS is low due to the high percentage of fiber, and in some cases the oil is a hindrance to animal digestion and lactating cow milk quality.

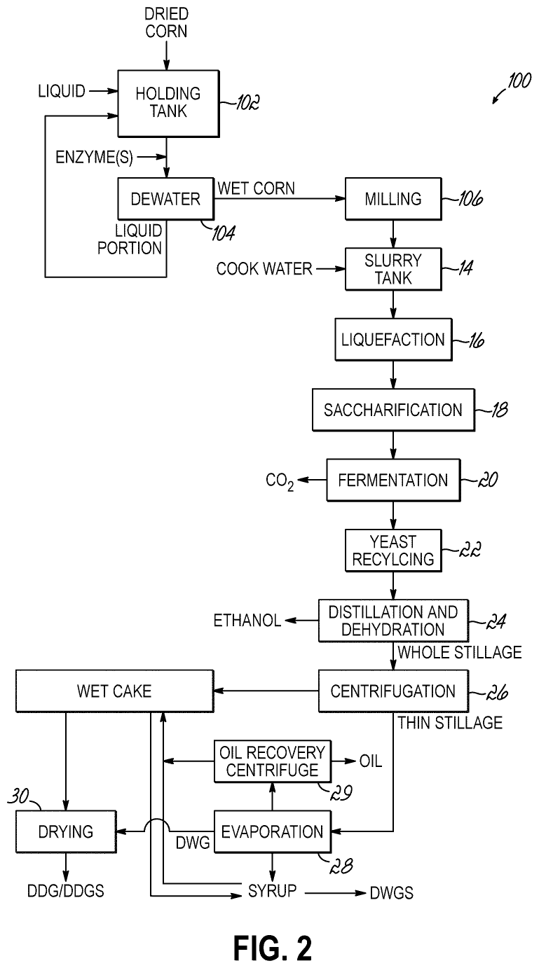

In accordance with the present invention, FIG. 2 shows one embodiment of a modified dry grind method and system for biochemical and/or biofuel production, such as alcohol (e.g., ethanol), collectively numeral 100, in which dried grain (e.g., corn) feedstock (or, alternatively, an already elevated moisture grain (e.g., corn) feedstock with a moisture content typically found in field harvested grain (e.g., 20-24% moisture or higher)) initially is wetted prior to being milled, and is based on the dry grind process 10 shown in FIG. 1 and described hereinabove, with modifications/improvements made thereto. This modified dry grind method and system 100 can reduce the amount of electricity typically consumed in the grain handling and milling part of a dry grind process, thereby helping to keep overall capital costs at a desirable level for the dry grind plant. The details of the modifications to FIG. 1 as set out in the embodiment of FIG. 2 are discussed hereinbelow. It is noted that certain reference numerals used in FIG. 1 are used here to represent like devices and/or steps in the method and system 100 of FIG. 2.

As shown in FIG. 2, in this embodiment, dried grain feedstock (e.g., corn) can be sent to a holding tank 102, which can include a continuous tank design, a plug flow tank design, a mixer to wet the corn prior to milling, and the like, whereat the corn can mix with a liquid and be contained in the holding tank 102 for about 30 seconds to about 10 hours to wet or moisten the grain (e.g., corn kernels) at a temperature of about 60° C. or less such as to avoid starch gelatinization. Higher temperatures can be utilized, which can be based on the contact time. Shorter contact times can utilize higher temperatures, such as 70° C., 80° C., or 90° C. In another example, the grain can be contained therein for 30 seconds to less than 4 hours. In another example, the grain can be contained therein for 30 seconds to 3 hours or for 30 seconds to 2 hours. In yet another example, the grain can be contained therein for 30 seconds to 30 minutes. In another example, the grain can be contained therein for 1, 2, 3, 4, 5, 6, 7, 8, 9, or 10 minutes and up to less than 4 hours. In still another example, the grain can be contained therein for 3 minutes to 30 minutes. In another example, the grain can be subjected to a temperature of about 55° C. or less. In another example, the grain can be subjected to a temperature of about 50° C. or less. In another example, the grain can be subjected to a temperature of about 65° C. In another example, the grain can be subjected to a temperature of about 70° C. The temperature of the mixture of grain and liquid in the holding tank 102 can be adjusted or controlled up or down, by means and methods known in the art, to a specified temperature or within a certain range(s) to ensure desirable wetting of the grain.

The liquid, which may be water, backset, and the like, can be supplied directly to the holding tank 102, as shown, or mixed with the grain feedstock prior to the holding tank 102, for example. In one example, the backset can include thin stillage from the back end of the method and system 100. The water may be fresh water and/or recycled from water in the method and system 100. The holding tank 102 can be appropriately sized to provide enough residence time for the grain to be desirably wetted. The holding tank 102 can be operated on level control, increasing or decreasing the amount of material moved or pumped out of the holding tank 102 to match the amount of material entering, by means and methods known in the art.

This wetting of the corn is understood to differ from traditional wet milling techniques in that a traditional wet mill plant is generally focused on softening/soaking the grain (e.g., corn), such as from 24 hours to 60 hours, so that the grain is soaked throughout the internal of the grain (e.g., corn kernel) whereby germ, for example, can be removed whole and intact in a first grind mill. In contrast, the wetting of the corn in the method and system 100 of FIG. 2 is not at all concerned with extracting whole germ or liquid/water migration into the internal of the kernel, so there is no need to soak the kernel for an extended period of time, such as up to 60 hours. In addition, the liquid in the holding tank 102 can be devoid of enzymes and other chemical aides, such as sodium sulfite, sulfur dioxide, or the like, that can assist with softening of the grain and the removal of whole germ. And thus, the grain here does not need to be subjected to the same.

After the holding tank 102, the mixture of wet corn and liquid (or already provided wet corn) may be subjected to enzyme and/or chemical treatment with one or more enzymes and/or chemicals to aid in starch reduction and/or facilitation of grain (e.g., whole kernel) breakage/milling of the grain. In another example, one or more enzymes and/or chemicals may be added directly to the holding tank 102 to aid in grain or kernel reduction and/or facilitation of grain (e.g., whole kernel) breakage/milling of the grain. In one example, the one or more enzymes can include amylase, alpha-amylase, glucoamylase, fungal, phytase, protease (e.g., serine protease), cellobiose, cellulase, hemicellulase, xylanase, glucanase, beta-glucanase, transglutaminase (e.g. microbial), and the like. In one example, the one or more chemicals can include sulfur dioxide, sodium sulfate, sodium chloride, hydrochloric acid, and the like.

After optionally being subjected to enzyme and/or chemical treatment and prior to being milled at milling step 106, the slurry mixture of wet corn and liquid can be subjected to an optional dewatering device 104 to remove a liquid portion (e.g., excess water) from the wet grain so that additional liquid is not subjected to milling, thereby further positively impacting or reducing the consumption of electricity. The separated liquid portion can be recycled back to the holding tank 102, as shown. The dewatering device 104 can include a screen, such as a gravity screen. In one example, the dewatering device 104 can include a paddle screen, pressure screen, and the like. The dewatering device 104 can include multiple dewatering devices, which can be utilized in series or parallel. The screen of the dewatering device 104 can be provided with openings desirably sized to separate the wet grain from the liquid portion. In one example, the screen openings can be of a size from 50 micron to 2,000 micron, but some plants can operate at less than or greater than these screen sizes.

From the holding tank 102 (or the optional dewatering device 104, if present), the wetted (and optionally enzymatically and/or chemically treated) corn then may be subjected to a milling step 106, which can include one or more disc mills, in which the wet grain (e.g., corn kernel) can be broken down or ground into smaller pieces or particles. In one example, the one or more disc mills may be replaced by a roller mill, a homogenizer, or the like. The disc mill, roller mill, homogenizer, and the like can be utilized in series or parallel. In one example, the disc mill can include a 36″ disc mill. In addition, it should be understood that the mill gap settings can be varied, as needed, to produce optimal settings, such as for the desired feedstock. The resulting particle distribution from the milling step 106 can yield a widely spread, bell type curve, which can include particle sizes as small as 45 microns and as large as 2 mm to 3 mm. The majority of the particles tend to be in the range of 500 to 2,000 microns, which is the “peak” of the bell curve.

Although not shown, additional aides to assist with preparation of the grain for milling or with grinding of the grain into smaller pieces can include, for example, sonication or ultrasound systems/devices and the like that can help in the grain (kernel) breakage. Such devices may be placed before or after the milling step 106. In addition and although not shown, the milled grain from milling step 106 can be further optionally subjected to a screening step prior to being mixed with cook water to create a slurry at the slurry tank 14 such as to help ensure a uniform/more desirable uniform particle size. The optional screening step can separate out and return still oversized grain particles back to the milling step 106 for further milling. The screening step can include a screening device with a screen, such as a gravity screen. In another example, the screening device can include a paddle screen, pressure screen, and the like. The screening device can include multiple screening devices, which can be utilized in series or parallel. The screen of the screening device can be provided with openings desirably sized to separate out any oversized grain particles. In one example, the screen openings can be of a size from 50 micron to 2,000 micron, but some plants can operate at less than or greater than these screen sizes. In another example, the screen openings can be from 150 to 450 micron. In another example, the screen openings can be 150, 250, or 450 micron. When more than one set of screens are used (e.g., paddle screens), the screens can have different size openings in each unit operation. For example, one screen can have openings of 250 micron while the other screen can have openings of 400 micron.

After the milling step 106 (or optional screening step), the milled grain can be further treated in accordance with that shown in FIG. 1 and described hereinabove, i.e., mixed with cook water to create a slurry at the slurry tank 14 whereat a commercial enzyme called alpha-amylase can be added (not shown) to produce biofuel and/or biochemicals, such as alcohol (e.g., ethanol). While the method and system 100 of FIG. 2 provides for the production of alcohol, such as ethanol, it should be understood that other alcohols, e.g., butanol, as well as other biofuels and/or biochemicals, can be produced here. In one example, other options for the sugar stream, aside from fermentation, can include further processing or refining of glucose to fructose or other simple or even complex carbohydrates, processing into feed, microbe-based fermentation (as opposed to yeast based) and other various chemical, pharmaceutical or nutraceutical processing (such as propanol, isobutanol, citric acid or succinic acid), and the like. Still further, the front end wet milling described in detail in FIG. 2 may be incorporated into other methods and systems that can produce additional or more desirable coproducts, along with DDG(S) and DWG(S).

As indicated above, the front end wet milling described in detail in FIG. 2 may be incorporated into any traditional, modified traditional, or non-traditional dry grind process/plant and by doing so can reduce the amount of electricity typically consumed in the grain handling and milling part of a dry grind process (by initially wetting the dried grain (e.g., corn) feedstock prior to milling the same), thereby helping to keep overall capital costs at a desirable level for the dry grind plant.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicant's general inventive concept.

What is claimed is:

1. A modified dry grind method for biochemical and/or biofuel production, comprising:
mixing a grain feedstock with a liquid in a tank for about 30 seconds to about 10 hours to wet the grain feedstock prior to any milling of the grain feedstock, wherein the mixture can be devoid of any enzyme and other chemical processing aides;
optionally dewatering the wetted grain feedstock to remove liquid therefrom;
milling the grain to break the grain into smaller ground pieces; and
thereafter, subjecting the milled grain to traditional or non-traditional dry grind steps to produce a biochemical and/or biofuel.
